(19) United States
(12) Patent Application Publication
(10) Pub. No.: US 2026/0290513 A1
(43) Pub. Date: Sep. 24, 2026

(54) SYSTEMS AND METHODS FOR CONFIGURING DATA IN A DISTRIBUTED NETWORKING ENVIRONMENT

(71) Applicant: BANK OF AMERICA CORPORATION, Charlotte, NC (US)

(72) Inventors: Rajalakshmi Arumugam, Chennai (IN); Thangaselvi Arichandrapandian, Chennai (IN); Aishwarya Srinivasan, Chennai (IN); Pavithraa Murugaiyan, Chennai (IN); Mathisurekha Muthuraj, Chennai (IN)

(73) Assignee: BANK OF AMERICA CORPORATION, Charlotte, NC (US)

(21) Appl. No.: 19/082,563

(22) Filed: Mar. 18, 2025

(51) Int. Cl. G16B 50/50; G06N 10/40; G16B 40/00

(52) U.S. Cl. CPC G16B 50/50; G06N 10/40; G16B 40/00

(57) ABSTRACT

Systems, computer program products, and methods are described herein for configuring data in a distributed networking environment. The present disclosure provides a system to receive an update of cached data from a source application associated with a source grid. The system may cluster the update and generate a unique identifier associated with the clustered update. The system may encode the clustered update using a digital DNA sequencing algorithm into a single digital DNA sequence. A source qubit may be generated based on the single digital DNA sequence. The source qubit may be entangled with a target qubit associated with a target grid. The single digital DNA sequence may be decoded, and the clustered update may be retrieved. The clustered update may be used to configure a target application's cache.

### TECHNOLOGICAL FIELD

Example embodiments of the present disclosure relate to configuring data in a distributed networking environment.

### BACKGROUND

There are significant issues associated with synchronization of cache updates in a distributed networking environment. Applicant has identified a number of deficiencies and problems associated with conventional methods for configuring data in a distributed networking environment. Through applied effort, ingenuity, and innovation, many of these identified problems have been solved by developing solutions that are included in embodiments of the present disclosure, many examples of which are described in detail herein.

### BRIEF SUMMARY

The following presents a simplified summary of one or more embodiments of the present disclosure, in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments of the present disclosure in a simplified form as a prelude to the more detailed description that is presented later.

Systems, methods, and computer program products are provided for configuring data in a distributed networking environment.

Embodiments of the present invention address the above needs and/or achieve other advantages by providing apparatuses (e.g., a system, computer program product, and/or other devices) and methods for configuring data in a distributed networking environment. The system embodiments may comprise a processing device and a non-transitory storage device containing instructions when executed by the processing device, to perform the steps disclosed herein. In computer program product embodiments of the invention, the computer program product comprises a non-transitory computer-readable medium comprising code causing an apparatus to perform the steps disclosed herein. Computer implemented method embodiments of the invention may comprise providing a computing system comprising a computer processing device and a non-transitory computer readable medium, where the computer readable medium comprises configured computer program instruction code, such that when said instruction code is operated by said computer processing device, said computer processing device performs certain operations to carry out the steps disclosed herein.

In some embodiments, the present disclosure provides a solution that may receive an update associated with cached data from a source application associated with a source grid. In some embodiments, the solution may cluster the update based on chronological order in which the update was received. In some embodiments, the solution may generate a unique identifier associated with the clustered update. In some embodiments, the solution may encode, via a digital DNA encoder, the clustered update using a digital DNA sequencing algorithm into a single digital DNA sequence. In some embodiments, the solution may generate, via a quantum platform, a source qubit based on the single digital DNA sequence. In some embodiments, the solution may configure the source qubit using a Greenberger-Horne-Zeilinger (GHZ) quantum multipartite entanglement technique to entangle the source qubit with a target qubit associated with a target grid. In some embodiments, the solution may decode, at the target grid and using a digital DNA decoder, the single digital DNA sequence received from the entangled qubits to retrieve the clustered update. In some embodiments, the solution may configured a target application's cached data with the clustered update.

In some embodiments, clustering the updated based on chronological order in which the updated was received includes configured the updates in chronological order for form the cluster, generating a unique identifier for the cluster, and assigning the unique identifier to the cluster.

In some embodiments, the target grid comprises at least one of the same components as the source grid.

In some embodiments, encoding the clustered update using the digital DNA sequencing algorithm may include generating the single digital DNA sequence using a digital DNA sequencing algorithm recommendation engine including a machine learning (ML) engine that recommends the digital DNA sequencing algorithm.

In some embodiments, the cached data may include multi-variant data including data types of at least vector data, unstructured data, key-value pair data, or multimedia data.

In some embodiments, a plurality of cached variant identifiers may be used to identify the data types of the cached data.

In some embodiments, encoding the clustered update using the digital DNA sequencing algorithm may include generating the single digital DNA sequencing using a digital DNA sequencing algorithm recommendation engine including a ML engine that recommends the digital DNA sequencing algorithm based on the plurality of cached variant identifiers.

In some embodiments, the digital DNA encoder may include error mitigation framework used to generate data that went missing during the encoding of the digital DNA sequence.

In some embodiments, the source qubit may include at least two sets of qubits including at least a first qubit associated with the update associated with the cached data from the source application, and at least a second qubit associated with the digital DNA sequencing algorithm.

The above summary is provided merely for purposes of summarizing some example embodiments to provide a basic understanding of some aspects of the present disclosure. Accordingly, it will be appreciated that the above-described embodiments are merely examples and should not be construed to narrow the scope or spirit of the disclosure in any way. It will be appreciated that the scope of the present disclosure encompasses many potential embodiments in addition to those here summarized, some of which will be further described below.

### BRIEF DESCRIPTION OF THE DRAWINGS

Having thus described embodiments of the disclosure in general terms, reference will now be made the accompanying drawings. The components illustrated in the figures may or may not be present in certain embodiments described herein. Some embodiments may include fewer (or more) components than those shown in the figures.

FIGS. 1A-1C illustrates technical components of an exemplary distributed computing environment for configuring data in a distributed networking environment, in accordance with an embodiment of the disclosure;

FIG. 2 illustrates an exemplary architecture of a machine learning (ML) subsystem 200, in accordance with an embodiment of the disclosure;

FIG. 3 illustrates a process flow for configuring data in a distributed networking environment, in accordance with an embodiment of the disclosure;

FIG. 4 illustrates an exemplary distributed networking environment including a source grid, a target grid, and a quantum platform, in accordance with an embodiment of the disclosure;

FIG. 5 illustrates an example of updating one or more grid applications of the target grid via cache coherence, in accordance with an embodiment of the disclosure; and

FIG. 6 illustrates the source grid updating, via the quantum platform, a first target grid, a second target grid, and an Nth target grid, in accordance with an embodiment of the disclosure.

### DETAILED DESCRIPTION

Embodiments of the present disclosure will now be described more fully hereinafter with reference to the accompanying drawings, in which some, but not all, embodiments of the disclosure are shown. Indeed, the disclosure may be embodied in many different forms and should not be construed as limited to the embodiments set forth herein; rather, these embodiments are provided so that this disclosure will satisfy applicable legal requirements. Where possible, any terms expressed in the singular form herein are meant to also include the plural form and vice versa, unless explicitly stated otherwise. Also, as used herein, the term “a” and/or “an” shall mean “one or more,” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on.” Like numbers refer to like elements throughout.

As used herein, an “entity” may be any institution employing information technology resources and particularly technology infrastructure configured for processing large amounts of data. Typically, these data can be related to the people who work for the organization, its products or services, the customers or any other aspect of the operations of the organization. As such, the entity may be any institution, group, association, financial institution, establishment, company, union, authority or the like, employing information technology resources for processing large amounts of data.

As described herein, a “user” may be an individual associated with an entity. As such, in some embodiments, the user may be an individual having past relationships, current relationships or potential future relationships with an entity. In some embodiments, the user may be an employee (e.g., an associate, a project manager, an IT specialist, a manager, an administrator, an internal operations analyst, or the like) of the entity or enterprises affiliated with the entity.

As used herein, a “user interface” may be a point of human-computer interaction and communication in a device that allows a user to input information, such as commands or data, into a device, or that allows the device to output information to the user. For example, the user interface includes a graphical user interface (GUI) or an interface to input computer-executable instructions that direct a processor to carry out specific functions. The user interface typically employs certain input and output devices such as a display, mouse, keyboard, button, touchpad, touch screen, microphone, speaker, LED, light, joystick, switch, buzzer, bell, and/or other user input/output device for communicating with one or more users.

As used herein, an “engine” may refer to core elements of an application, or part of an application that serves as a foundation for a larger piece of software and drives the functionality of the software. In some embodiments, an engine may be self-contained, but externally-controllable code that encapsulates powerful logic designed to perform or execute a specific type of function. In one aspect, an engine may be underlying source code that establishes file hierarchy, input and output methods, and how a specific part of an application interacts or communicates with other software and/or hardware. The specific components of an engine may vary based on the needs of the specific application as part of the larger piece of software. In some embodiments, an engine may be configured to retrieve resources created in other applications, which may then be ported into the engine for use during specific operational aspects of the engine. An engine may be configurable to be implemented within any general purpose computing system. In doing so, the engine may be configured to execute source code embedded therein to control specific features of the general purpose computing system to execute specific computing operations, thereby transforming the general purpose system into a specific purpose computing system.

As used herein, “authentication credentials” may be any information that can be used to identify of a user. For example, a system may prompt a user to enter authentication information such as a username, a password, a personal identification number (PIN), a passcode, biometric information (e.g., iris recognition, retina scans, fingerprints, finger veins, palm veins, palm prints, digital bone anatomy/structure and positioning (distal phalanges, intermediate phalanges, proximal phalanges, and the like), an answer to a security question, a unique intrinsic user activity, such as making a predefined motion with a user device. This authentication information may be used to authenticate the identity of the user (e.g., determine that the authentication information is associated with the account) and determine that the user has authority to access an account or system. In some embodiments, the system may be owned or operated by an entity. In such embodiments, the entity may employ additional computer systems, such as authentication servers, to validate and certify resources inputted by the plurality of users within the system. The system may further use its authentication servers to certify the identity of users of the system, such that other users may verify the identity of the certified users. In some embodiments, the entity may certify the identity of the users. Furthermore, authentication information or permission may be assigned to or required from a user, application, computing node, computing cluster, or the like to access stored data within at least a portion of the system.

It should also be understood that “operatively coupled,” as used herein, means that the components may be formed integrally with each other, or may be formed separately and coupled together. Furthermore, “operatively coupled” means that the components may be formed directly to each other, or to each other with one or more components located between the components that are operatively coupled together. Furthermore, “operatively coupled” may mean that the components are detachable from each other, or that they are permanently coupled together. Furthermore, operatively coupled components may mean that the components retain at least some freedom of movement in one or more directions or may be rotated about an axis (i.e., rotationally coupled, pivotally coupled). Furthermore, “operatively coupled” may mean that components may be electronically connected and/or in fluid communication with one another.

As used herein, an “interaction” may refer to any communication between one or more users, one or more entities or institutions, one or more devices, nodes, clusters, or systems within the distributed computing environment described herein. For example, an interaction may refer to a transfer of data between devices, an accessing of stored data by one or more nodes of a computing cluster, a transmission of a requested task, or the like.

It should be understood that the word “exemplary” is used herein to mean “serving as an example, instance, or illustration.” Any implementation described herein as “exemplary” is not necessarily to be construed as advantageous over other implementations.

As used herein, “determining” may encompass a variety of actions. For example, “determining” may include calculating, computing, processing, deriving, investigating, ascertaining, and/or the like. Furthermore, “determining” may also include receiving (e.g., receiving information), accessing (e.g., accessing data in a memory), and/or the like. Also, “determining” may include resolving, selecting, choosing, calculating, establishing, and/or the like. Determining may also include ascertaining that a parameter matches a predetermined criterion, including that a threshold has been met, passed, exceeded, and so on.

In the modern computing world, there are significant issues associated with updating caches of various applications. In some cases, a source application may be used to update a target application. In this way, the cached data of the source application may be transmitted to the cache of the target application to update it. With conventional methods, however, the synching up of cache updates across multiple applications in a multi grid computing platform is a resource intensive process involving multiple parties and associated cache products. Further, using multi-variant caches such as vector, un-structed, key-value pair, and multi-media data across applications in various grids make conventional cache coherence processes complex in nature. In addition, traditional cache systems require communication over networks which introduces additional latency and network overhead, which may increase with the number of nodes in the cache cluster and the frequency of cache accesses. The increase in latency and overhead negates the performance gains from such a system, especially for frequently accessed data. Further, there are scalability issues associated with conventional distributed cache systems. Factors such as the overhead of coordinating cache operations, network congestion, or contention for shared resources can limit the scalability of the system beyond a certain point. Additionally, a conventional distributed cache system introduces potential entry points for security vulnerabilities. To address these concerns, systems and methods for configuring data in a distributed networking environment are introduced.

The solutions as described here includes a hybrid technique that may capture a cluster of multivariant data of cache updates from various applications and convert the cache data into a digital DNA sequence, which is then transformed into quantum qubits. The qubits are used for instant coherence of the cache changes in all corresponding grid systems in the distributed network. In this way, an application associated with a source grid may store an update in its cache. A clustering engine may cluster one or more cache updates, depending on the file type of the update, into a clustered update. The clustered update may be encoded into a single digital DNA sequence by a digital DNA encoder. The digital DNA sequence encoder may use a digital DNA sequence algorithm recommendation engine that uses deep learning techniques to choose the best algorithm for converting the cache data into the DNA sequence. The digital DNA sequence may then be transformed into qubits using, for example, LUBY transformation techniques. The qubits may include qubits that correspond to the cache data and to the DNA sequencing algorithm used. The source qubits may be entangled with qubits associated with a target grid using the Greenberger-Horne-Zeilinger (GHZ) entanglement technique. The target grid may then decode the digital DNA sequence and convert the digital DNA sequence into data to be used in a target application's cache. The target application's cache may then be updated to reflect the update from the source application's cache update.

What is more, the present disclosure provides a technical solution to a technical problem. As described herein, the technical problem includes the resource-intensive, complex, and error-prone process of synchronizing cache updates across multiple applications in multi-grid computing environments—particularly when handling multi-variant data types and security concerns. The technical solution presented herein allows for using digital DNA encoding and quantum entanglement techniques to efficiently cluster and transmit cache updates across distributed grids. In particular, the solution as described herein replaces traditional multi-step, networking intensive processes with a hybrid quantum-based synchronization process that reduces latency and overhead and improves data security. The solution as described herein is an improvement over existing solutions to the above-described synchronization problems, (i) with fewer steps to achieve the solution, thus reducing the amount of computing resources, such as processing resources, storage resources, network resources, and/or the like, that are being used (e.g., eliminating large-scale multicast or repeated fetch processes for each cache update), (ii) providing a more accurate solution to problem, thus reducing the number of resources required to remedy any errors made due to a less accurate solution (e.g., automatically identifying the optimal digital DNA sequence through deep learning to reduce manual corrective steps), (iii) removing manual input and waste from the implementation of the solution, thus improving speed and efficiency of the process and conserving computing resources (e.g., removing manual configuration of caching protocols for each file and/or data type), (iv) determining an optimal amount of resources that need to be used to implement the solution, thus reducing network traffic and load on existing computing resources (e.g., skipping updates for rarely accessed data). Furthermore, the technical solution described herein uses a rigorous, computerized process to perform specific tasks and/or activities that were not previously performed. In specific implementations, the technical solution bypasses a series of steps previously implemented, thus further conserving computing resources.

In addition, the technical solution described herein is an improvement to computer technology and is directed to non-abstract improvements to the functionality of a computer platform itself. Specifically, the solution as described herein is a solution to the problem of resource-intensive, error-prone synchronization of multi-variant cache updates across distributed grids. Further, the solutions as described herein may be characterized as identifying a specific improvement in computer capabilities and/or network functionalities in response to the solution's integration to existing devices, software, applications, and/or the like. In this way, the solution improves the capability of a system to efficiently cluster and convert cache data into quantum-entangled digital DNA sequences for instantaneous coherence of updates across multiple grids. Further, the solution improves the functionality of networks in response to reducing the resources consumed by the system (e.g., network resources, computing resources, memory resources, and/or the like).

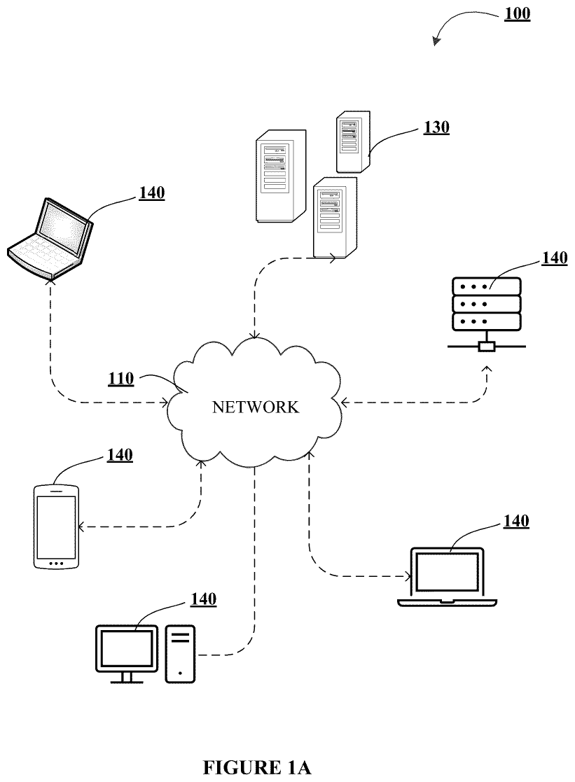

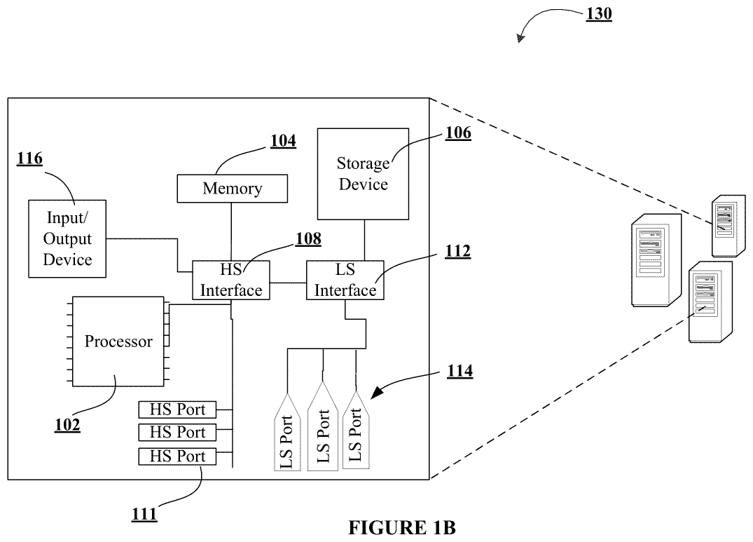

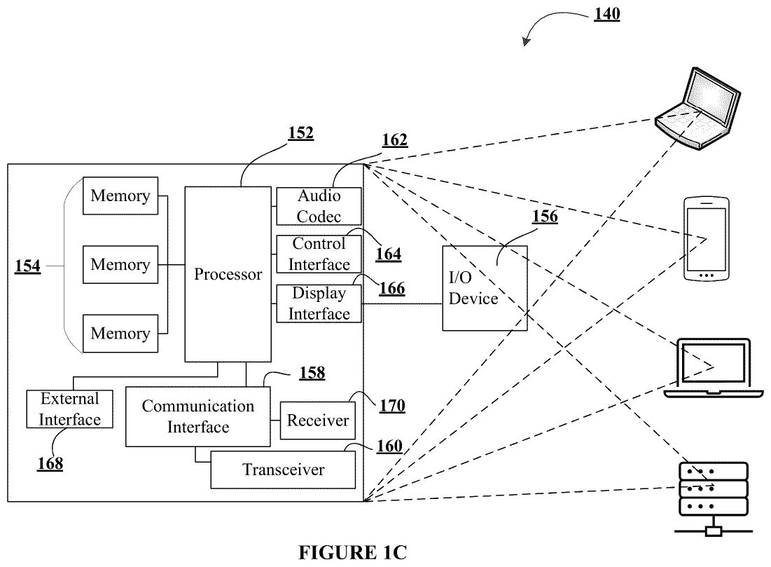

FIGS. 1A-1C illustrate technical components of an exemplary distributed computing environment 100 for configuring data in a distributed networking environment, in accordance with an embodiment of the disclosure. As shown in FIG. 1A, the distributed computing environment 100 contemplated herein may include a system 130, an end-point device(s) 140, and a network 110 over which the system 130 and end-point device(s) 140 communicate therebetween. FIG. 1A illustrates only one example of an embodiment of the distributed computing environment 100, and it will be appreciated that in other embodiments one or more of the systems, devices, and/or servers may be combined into a single system, device, or server, or be made up of multiple systems, devices, or servers. Also, the distributed computing environment 100 may include multiple systems, same or similar to system 130, with each system providing portions of the necessary operations (e.g., as a server bank, a group of blade servers, or a multi-processor system).

In some embodiments, the system 130 and the end-point device(s) 140 may have a client-server relationship in which the end-point device(s) 140 are remote devices that request and receive service from a centralized server (e.g., system 130). In some other embodiments, the system 130 and the end-point device(s) 140 may have a peer-to-peer relationship in which the system 130 and the end-point device(s) 140 are considered equal and all have the same abilities to use the resources available on the network 110. Instead of having a central server (e.g., system 130) which would act as the shared drive, each device that is connect to the network 110 would act as the server for the files stored on it.

The system 130 may represent various forms of servers, such as web servers, database servers, file server, or the like, various forms of digital computing devices, such as laptops, desktops, video recorders, audio/video players, radios, workstations, or the like, or any other auxiliary network devices, such as wearable devices, Internet-of-things devices, electronic kiosk devices, mainframes, or the like, or any combination of the aforementioned.

The end-point device(s) 140 may represent various forms of electronic devices, including user input devices such as personal digital assistants, cellular telephones, smartphones, laptops, desktops, and/or the like, merchant input devices such as point-of-sale (POS) devices, electronic payment kiosks, resource distribution devices, and/or the like, electronic telecommunications device (e.g., automated teller machine (ATM)), and/or edge devices such as routers, routing switches, integrated access devices (IAD), and/or the like.

The network 110 may be a distributed network that is spread over different networks. This provides a single data communication network, which can be managed jointly or separately by each network. Besides shared communication within the network, the distributed network often also supports distributed processing. In some embodiments, the network 110 may include a telecommunication network, local area network (LAN), a wide area network (WAN), and/or a global area network (GAN), such as the Internet. Additionally, or alternatively, the network 110 may be secure and/or unsecure and may also include wireless and/or wired and/or optical interconnection technology. The network 110 may include one or more wired and/or wireless networks. For example, the network 110 may include a cellular network (e.g., a long-term evolution (LTE) network, a code division multiple access (CDMA) network, a 3G network, a 4G network, a 5G network, another type of next generation network, and/or the like), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, a cloud computing network, or the like, and/or a combination of these or other types of networks.

It is to be understood that the structure of the distributed computing environment and its components, connections and relationships, and their functions, are meant to be exemplary only, and are not meant to limit implementations of the disclosures described and/or claimed in this document. In one example, the distributed computing environment 100 may include more, fewer, or different components. In another example, some or all of the portions of the distributed computing environment 100 may be combined into a single portion, or all of the portions of the system 130 may be separated into two or more distinct portions.

FIG. 1B illustrates an exemplary component-level structure of the system 130, in accordance with an embodiment of the disclosure. As shown in FIG. 1B, the system 130 may include a processor 102, memory 104, storage device 106, a high-speed interface 108 connecting to memory 104, high-speed expansion points 111, and a low-speed interface 112 connecting to a low-speed bus 114, and an input/output (I/O) device 116. The system 130 may also include a high-speed interface 108 connecting to the memory 104, and a low-speed interface 112 connecting to low-speed port 114 and storage device 106. Each of the components 102, 104, 106, 108, 111, and 112 may be operatively coupled to one another using various buses and may be mounted on a common motherboard or in other manners as appropriate. As described herein, the processor 102 may include a number of subsystems to execute the portions of processes described herein. Each subsystem may be a self-contained component of a larger system (e.g., system 130) and capable of being configured to execute specialized processes as part of the larger system. The processor 102 may process instructions for execution within the system 130, including instructions stored in the memory 104 and/or on the storage device 106 to display graphical information for a GUI on an external input/output device, such as a display 116 coupled to a high-speed interface 108. In some embodiments, multiple processors, multiple buses, multiple memories, multiple types of memory, and/or the like may be used. Also, multiple systems, same or similar to system 130, may be connected, with each system providing portions of the necessary operations (e.g., as a server bank, a group of blade servers, a multi-processor system, and/or the like). In some embodiments, the system 130 may be managed by an entity, such as a business, a merchant, a financial institution, a card management institution, a software and/or hardware development company, a software and/or hardware testing company, and/or the like. The system 130 may be located at a facility associated with the entity and/or remotely from the facility associated with the entity.

The processor 102 can process instructions, such as instructions of an application that may perform the functions disclosed herein. These instructions may be stored in the memory 104 (e.g., non-transitory storage device) or on the storage device 106, for execution within the system 130 using any subsystems described herein. It is to be understood that the system 130 may use, as appropriate, multiple processors, along with multiple memories, and/or I/O devices, to execute the processes described herein.

The memory 104 may store information within the system 130. In one implementation, the memory 104 is a volatile memory unit or units, such as volatile random access memory (RAM) having a cache area for the temporary storage of information, such as a command, a current operating state of the distributed computing environment 100, an intended operating state of the distributed computing environment 100, instructions related to various methods and/or functionalities described herein, and/or the like. In another implementation, the memory 104 is a non-volatile memory unit or units. The memory 104 may also be another form of computer-readable medium, such as a magnetic or optical disk, which may be embedded and/or may be removable. The non-volatile memory may additionally or alternatively include an EEPROM, flash memory, and/or the like for storage of information such as instructions and/or data that may be read during execution of computer instructions. The memory 104 may store, recall, receive, transmit, and/or access various files and/or information used by the system 130 during operation. The memory 104 may store any one or more of pieces of information and data used by the system in which it resides to implement the functions of that system. In this regard, the system may dynamically utilize the volatile memory over the non-volatile memory by storing multiple pieces of information in the volatile memory, thereby reducing the load on the system and increasing the processing speed.

The storage device 106 is capable of providing mass storage for the system 130. In one aspect, the storage device 106 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier may be a non-transitory computer-or machine-readable storage medium, such as the memory 104, the storage device 106, or memory on processor 102.

In some embodiments, the system 130 may be configured to access, via the network 110, a number of other computing devices (not shown). In this regard, the system 130 may be configured to access one or more storage devices and/or one or more memory devices associated with each of the other computing devices. In this way, the system 130 may implement dynamic allocation and de-allocation of local memory resources among multiple computing devices in a parallel and/or distributed system. Given a group of computing devices and a collection of interconnected local memory devices, the fragmentation of memory resources is rendered irrelevant by configuring the system 130 to dynamically allocate memory based on availability of memory either locally, or in any of the other computing devices accessible via the network. In effect, the memory may appear to be allocated from a central pool of memory, even though the memory space may be distributed throughout the system. Such a method of dynamically allocating memory provides increased flexibility when the data size changes during the lifetime of an application and allows memory reuse for better utilization of the memory resources when the data sizes are large.

The high-speed interface 108 manages bandwidth-intensive operations for the system 130, while the low-speed interface 112 manages lower bandwidth-intensive operations. Such allocation of functions is exemplary only. In some embodiments, the high-speed interface 108 is coupled to memory 104, input/output (I/O) device 116 (e.g., through a graphics processor or accelerator), and to high-speed expansion ports 111, which may accept various expansion cards (not shown). In such an implementation, low-speed interface 112 is coupled to storage device 106 and low-speed expansion port 114. The low-speed expansion port 114, which may include various communication ports (e.g., USB, Bluetooth, Ethernet, wireless Ethernet), may be coupled to one or more input/output devices, such as a keyboard, a pointing device, a scanner, or a networking device such as a switch or router (e.g., through a network adapter).

The system 130 may be implemented in a number of different forms. For example, the system 130 may be implemented as a standard server, or multiple times in a group of such servers. Additionally, the system 130 may also be implemented as part of a rack server system or a personal computer (e.g., laptop computer, desktop computer, tablet computer, mobile telephone, and/or the like). Alternatively, components from system 130 may be combined with one or more other same or similar systems and an entire system 130 may be made up of multiple computing devices communicating with each other.

FIG. 1C illustrates an exemplary component-level structure of the end-point device(s) 140, in accordance with an embodiment of the disclosure. As shown in FIG. 1C, the end-point device(s) 140 includes a processor 152, memory 154, an input/output device such as a display 156, a communication interface 158, and a transceiver 160, among other components. The end-point device(s) 140 may also be provided with a storage device, such as a microdrive or other device, to provide additional storage. Each of the components 152, 154, 156, 158, 160, 162, 164, 166, 168 and 170, are interconnected using various buses, and several of the components may be mounted on a common motherboard or in other manners as appropriate.

The processor 152 is configured to execute instructions within the end-point device(s) 140, including instructions stored in the memory 154, which in one embodiment includes the instructions of an application that may perform the functions disclosed herein, including certain logic, data processing, and data storing functions. The processor 152 may be implemented as a chipset of chips that include separate and multiple analog and digital processors. The processor 152 may be configured to provide, for example, for coordination of the other components of the end-point device(s) 140, such as control of user interfaces, applications run by end-point device(s) 140, and wireless communication by end-point device(s) 140.

The processor 152 may be configured to communicate with the user through control interface 164 and display interface 166 coupled to a display 156 (e.g., input/output device 156). The display 156 may be, for example, a Thin-Film-Transistor Liquid Crystal Display (TFT LCD) or an Organic Light Emitting Diode (OLED) display, or other appropriate display technology. An interface of the display may include appropriate circuitry and configured for driving the display 156 to present graphical and other information to a user. The control interface 164 may receive commands from a user and convert them for submission to the processor 152. In addition, an external interface 168 may be provided in communication with processor 152, so as to enable near area communication of end-point device(s) 140 with other devices. External interface 168 may provide, for example, for wired communication in some implementations, or for wireless communication in other implementations, and multiple interfaces may also be used.

The memory 154 stores information within the end-point device(s) 140. The memory 154 can be implemented as one or more of a computer-readable medium or media, a volatile memory unit or units, or a non-volatile memory unit or units. Expansion memory may also be provided and connected to end-point device(s) 140 through an expansion interface (not shown), which may include, for example, a Single In Line Memory Module (SIMM) card interface. Such expansion memory may provide extra storage space for end-point device(s) 140 or may also store applications or other information therein. In some embodiments, expansion memory may include instructions to carry out or supplement the processes described above and may include secure information also. For example, expansion memory may be provided as a security module for end-point device(s) 140 and may be programmed with instructions that permit secure use of end-point device(s) 140. In addition, secure applications may be provided via the SIMM cards, along with additional information, such as placing identifying information on the SIMM card in a non-hackable manner. In some embodiments, the user may use applications to execute processes described with respect to the process flows described herein. For example, one or more applications may execute the process flows described herein. In some embodiments, one or more applications stored in the system 130 and/or the user input system 140 may interact with one another and may be configured to implement any one or more portions of the various user interfaces and/or process flow described herein.

The memory 154 may include, for example, flash memory and/or NVRAM memory. In one aspect, a computer program product is tangibly embodied in an information carrier. The computer program product contains instructions that, when executed, perform one or more methods, such as those described herein. The information carrier is a computer-or machine-readable medium, such as the memory 154, expansion memory, memory on processor 152, or a propagated signal that may be received, for example, over transceiver 160 or external interface 168.

In some embodiments, the user may use the end-point device(s) 140 to transmit and/or receive information or commands to and from the system 130 via the network 110. Any communication between the system 130 and the end-point device(s) 140 may be subject to an authentication protocol allowing the system 130 to maintain security by permitting only authenticated users (or processes) to access the protected resources of the system 130, which may include servers, databases, applications, and/or any of the components described herein. To this end, the system 130 may trigger an authentication subsystem that may require the user (or process) to provide authentication credentials to determine whether the user (or process) is eligible to access the protected resources. Once the authentication credentials are validated and the user (or process) is authenticated, the authentication subsystem may provide the user (or process) with permissioned access to the protected resources. Similarly, the end-point device(s) 140 may provide the system 130 (or other client devices) permissioned access to the protected resources of the end-point device(s) 140, which may include a GPS device, an image capturing component (e.g., camera), a microphone, and/or a speaker.

The end-point device(s) 140 may communicate with the system 130 through communication interface 158, which may include digital signal processing circuitry where necessary. Communication interface 158 may provide for communications under various modes or protocols, such as GSM voice calls, SMS, EMS, or MMS messaging, CDMA, TDMA, PDC, WCDMA, CDMA2000, GPRS, and/or the like. Such communication may occur, for example, through transceiver 160. Additionally, or alternatively, short-range communication may occur, such as using a Bluetooth, Wi-Fi, near-field communication (NFC), and/or other such transceiver (not shown). Additionally, or alternatively, a Global Positioning System (GPS) receiver module 170 may provide additional navigation-related and/or location-related wireless data to user input system 140, which may be used as appropriate by applications running thereon, and in some embodiments, one or more applications operating on the system 130.

Communication interface 158 may provide for communications under various modes or protocols, such as the Internet Protocol (IP) suite (commonly known as TCP/IP). Protocols in the IP suite define end-to-end data handling methods for everything from packetizing, addressing and routing, to receiving. Broken down into layers, the IP suite includes the link layer, containing communication methods for data that remains within a single network segment (link); the Internet layer, providing internetworking between independent networks; the transport layer, handling host-to-host communication; and the application layer, providing process-to-process data exchange for applications. Each layer contains a stack of protocols used for communications.

The end-point device(s) 140 may also communicate audibly using audio codec 162, which may receive spoken information from a user and convert the spoken information to usable digital information. Audio codec 162 may likewise generate audible sound for a user, such as through a speaker, e.g., in a handset of end-point device(s) 140. Such sound may include sound from voice telephone calls, may include recorded sound (e.g., voice messages, music files, etc.) and may also include sound generated by one or more applications operating on the end-point device(s) 140, and in some embodiments, one or more applications operating on the system 130.

Various implementations of the distributed computing environment 100, including the system 130 and end-point device(s) 140, and techniques described here can be realized in digital electronic circuitry, integrated circuitry, specially designed application specific integrated circuits (ASICs), computer hardware, firmware, software, and/or combinations thereof.

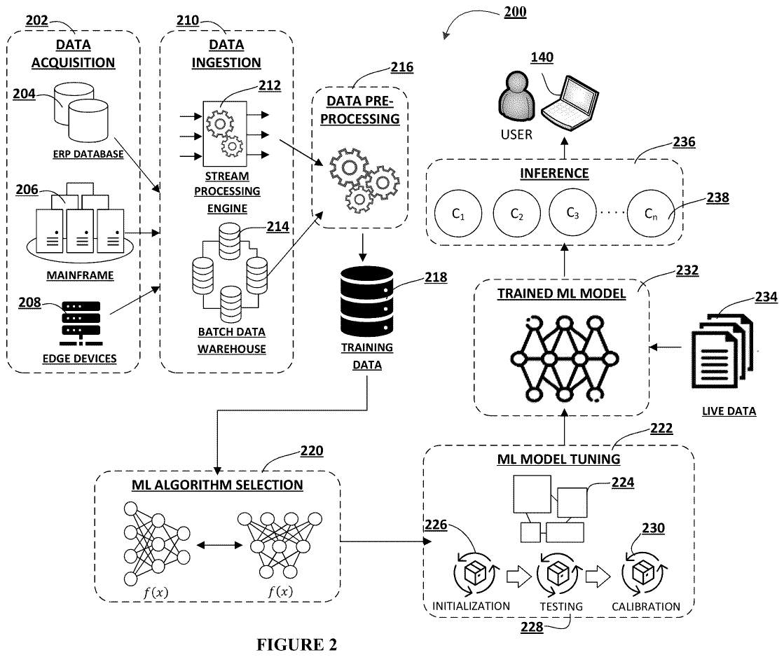

FIG. 2 illustrates an exemplary architecture of a machine learning (ML) subsystem 200, in accordance with an embodiment of the disclosure. The machine learning subsystem 200 may include a data acquisition engine 202, data ingestion engine 210, data pre-processing engine 216, ML model tuning engine 222, and inference engine 236.

The data acquisition engine 202 may identify various internal and/or external data sources to generate, test, and/or integrate new features for training the machine learning model 224. These internal and/or external data sources 204, 206, and 208 may be initial locations where the data originates or where physical information is first digitized. The data acquisition engine 202 may identify the location of the data and may describe connection characteristics for access and retrieval of data. In some embodiments, data is transported from each data source 204, 206, or 208 using any applicable network protocols, such as the File Transfer Protocol (FTP), Hyper-Text Transfer Protocol (HTTP), or any of the myriad of Application Programming Interfaces (APIs) provided by websites, networked applications, and other services. In some embodiments, the these data sources 204, 206, and 208 may include Enterprise Resource Planning (ERP) databases that host data related to day-to-day business activities such as accounting, procurement, project management, exposure management, supply chain operations, and/or the like, mainframes that are often the entity's central data processing center, edge devices that may be any piece of hardware, such as sensors, actuators, gadgets, appliances, or machines, that may be programmed for certain applications and may transmit data over the internet or other networks, and/or the like. The data acquired by the data acquisition engine 202 from these data sources 204, 206, and 208 may then be transported to the data ingestion engine 210 for further processing.

Depending on the nature of the data imported from the data acquisition engine 202, the data ingestion engine 210 may move the data to a destination for storage or further analysis. Typically, the data imported from the data acquisition engine 202 may be in varying formats as they come from different sources, including RDBMS, other types of databases, S3 buckets, CSVs, or from streams. Since the data comes from different locations, the data may need to be cleansed and transformed so that it may be analyzed together with data from other sources. At the data ingestion engine 210, the data may be ingested in real-time, using the stream processing engine 212, in batches using the batch data warehouse 214, or in a combination of both. The stream processing engine 212 may be used to process continuous data streams (e.g., data from edge devices) by computing on data directly as it is received, and filtering the incoming data to retain specific portions that are deemed useful by aggregating, analyzing, transforming, and/or ingesting the data. On the other hand, the batch data warehouse 214 may collect and transfer data in batches according to scheduled intervals, triggered events, and/or any other logical ordering.

In machine learning, the quality of data and the useful information that may be derived therefrom, directly affects the ability of the machine learning model 224 to learn. The data pre-processing engine 216 may implement advanced integration and processing steps needed to prepare the data for machine learning execution. This may include modules to perform any upfront data transformations to consolidate the data into alternate forms by changing the value, structure, and/or format of the data by using generalization, normalization, attribute selection, and aggregation, to data clean by filling missing values, smoothing noisy data, resolving inconsistent data, removing outliers, and/or any other encoding steps as needed.

In addition to improving the quality of the data, the data pre-processing engine 216 may implement feature extraction and/or selection techniques to generate training data 218. Feature extraction and/or selection is a process of transforming and/or reducing the data into new features that may better represent underlying patterns in the data. Additionally, or alternatively, feature extraction and/or selection may be a process of dimensionality reduction by which an initial set of data is reduced to more manageable groups for processing. A characteristic of these large data sets is a large number of variables that require a lot of computing resources to process. Feature extraction and/or selection may be used to select and/or combine variables into features, effectively reducing the amount of data that must be processed, while still accurately and completely describing the original data set. Depending on the type of machine learning algorithm being used, this training data 218 may require further enrichment. For example, in supervised learning, the training data may be enriched using one or more meaningful and informative labels to provide context such that a machine learning model may learn from the provided context. For example, labels may indicate whether a photo contains a bird or a car, which words were uttered in an audio recording, or if an x-ray contains a tumor. Data labeling is required for a variety of use cases including computer vision, natural language processing, and speech recognition. In contrast, unsupervised learning may use unlabeled data to find patterns in the data, such as inferences or clustering of data points.

The ML model tuning engine 222 may be used to train a machine learning model 224 using the training data 218 to make predictions or decisions without explicitly being programmed to do so. The machine learning model 224 represents what was learned by the selected machine learning algorithm 220 and represents the rules, numbers, and any other algorithm-specific data structures required for classification. Selecting the right machine learning algorithm may depend on a number of different factors, such as the problem statement and the kind of output needed, the type and the size of the data, the available computational time, the number of features and observations in the data, and/or the like. Machine learning algorithms may refer to programs (e.g., math and logic) that may be configured to self-adjust and perform better as they are exposed to more data. To this extent, machine learning algorithms are capable of adjusting their own parameters, given feedback on previous performance in making prediction about a dataset.

The machine learning algorithms contemplated, described, and/or used herein include supervised learning (e.g., using logistic regression, using back propagation neural networks, using random forests, decision trees, or the like), unsupervised learning (e.g., using an Apriori algorithm, using K-means clustering), semi-supervised learning, reinforcement learning (e.g., using a Q-learning algorithm, using temporal difference learning), and/or any other suitable machine learning model type. Each of these types of machine learning algorithms can implement any of one or more of a regression algorithm (e.g., ordinary least squares, logistic regression, stepwise regression, multivariate adaptive regression splines, locally estimated scatterplot smoothing, or the like), an instance-based method (e.g., k-nearest neighbor, learning vector quantization, self-organizing map, or the like), a regularization method (e.g., ridge regression, least absolute shrinkage and selection operator, elastic net, or the like), a decision tree learning method (e.g., classification and regression tree, iterative dichotomiser 3, C4.5, chi-squared automatic interaction detection, decision stump, random forest, multivariate adaptive regression splines, gradient boosting machines, or the like), a Bayesian method (e.g., naïve Bayes, averaged one-dependence estimators, Bayesian belief network, or the like), a kernel method (e.g., a support vector machine, a radial basis function, or the like), a clustering method (e.g., k-means clustering, expectation maximization, or the like), an associated rule learning algorithm (e.g., an Apriori algorithm, an Eclat algorithm, or the like), an artificial neural network model (e.g., a Perceptron method, a back-propagation method, a Hopfield network method, a self-organizing map method, a learning vector quantization method, or the like), a deep learning algorithm (e.g., a restricted Boltzmann machine, a deep belief network method, a convolution network method, a stacked auto-encoder method, or the like), a dimensionality reduction method (e.g., principal component analysis, partial least squares regression, Sammon mapping, multidimensional scaling, projection pursuit, or the like), an ensemble method (e.g., boosting, bootstrapped aggregation, AdaBoost, stacked generalization, gradient boosting machine method, random forest method, or the like), and/or the like.

To tune the machine learning model, the ML model tuning engine 222 may repeatedly execute cycles of experimentation including initialization 226, testing 228, and/or calibration 230 to optimize the performance of the machine learning algorithm 220 and refine the results in preparation for deployment of those results for consumption or decision making. To this end, the ML model tuning engine 222 may dynamically vary hyperparameters each iteration (e.g., number of trees in a tree-based algorithm or the value of alpha in a linear algorithm), run the algorithm on the data again, then compare its performance on a validation set to determine which set of hyperparameters results in the most accurate model. The accuracy of the model is the measurement used to determine which set of hyperparameters is best at identifying relationships and patterns between variables in a dataset based on the input, or training data 218. A fully trained machine learning model 232 is one whose hyperparameters are tuned and whose model accuracy is maximized.

The trained machine learning model 232, similar to any other software application output, may be persisted to storage, file, memory, or application, or looped back into the processing component to be reprocessed. More often, the trained machine learning model 232 is deployed into an existing production environment to make practical business decisions based on live data 234. To this end, the machine learning subsystem 200 uses the inference engine 236 to make such decisions. The type of decision-making may depend upon the type of machine learning algorithm used. For example, machine learning models trained using supervised learning algorithms may be used to structure computations in terms of categorized outputs (e.g., C₁, C₂, . . . , Cₙ 238) or observations based on defined classifications, represent possible solutions to a decision based on certain conditions, model complex relationships between inputs and outputs to find patterns in data or capture a statistical structure among variables with unknown relationships, and/or the like. On the other hand, machine learning models trained using unsupervised learning algorithms may be used to group (e.g., C₁, C₂, . . . , Cₙ 238) live data 234 based on how similar they are to one another to solve exploratory challenges where little may be known about the data, provide a description or label (e.g., C₁, C₂, . . . , Cₙ 238) to live data 234, such as in classification, and/or the like. These categorized outputs, groups (clusters), or labels may then be presented to the user input system 140. In still other cases, machine learning models that perform regression techniques may use live data 234 to predict or forecast continuous outcomes.

It will be understood that the embodiment of the machine learning subsystem 200 illustrated in FIG. 2 is exemplary and that other embodiments may vary. As another example, in some embodiments, the machine learning subsystem 200 may include more, fewer, or different components.

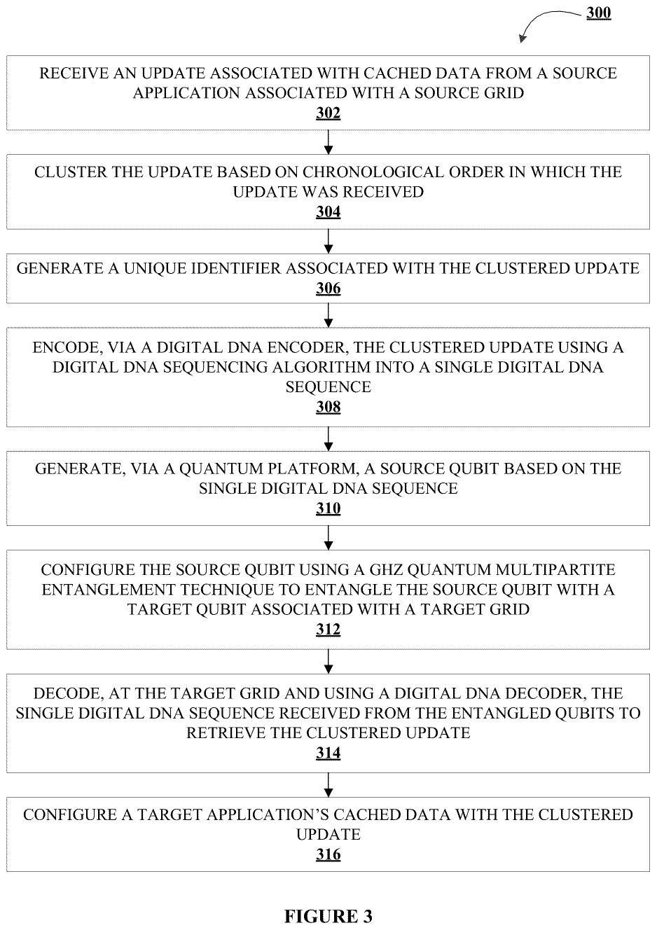

FIG. 3 illustrates a process flow for configuring data in a distributed networking environment, in accordance with an embodiment of the disclosure. The method may be carried out by various components of the distributed computing environment 100 discussed herein (e.g., the system 130, one or more end-point device(s) 140, etc.). An example system may include at least one processing device and at least one non-transitory storage device with computer-readable program code stored thereon and accessible by the at least one processing device, wherein the computer-readable code when executed is configured to carry out the method discussed herein.

In some embodiments, the system (e.g., similar to one or more of the systems described herein with respect to FIGS. 1A-1C) may perform one or more of the steps of process flow 300. For example, the system (e.g., the system 130 described herein with respect to FIGS. 1A-1C) may perform the steps of process flow 300.

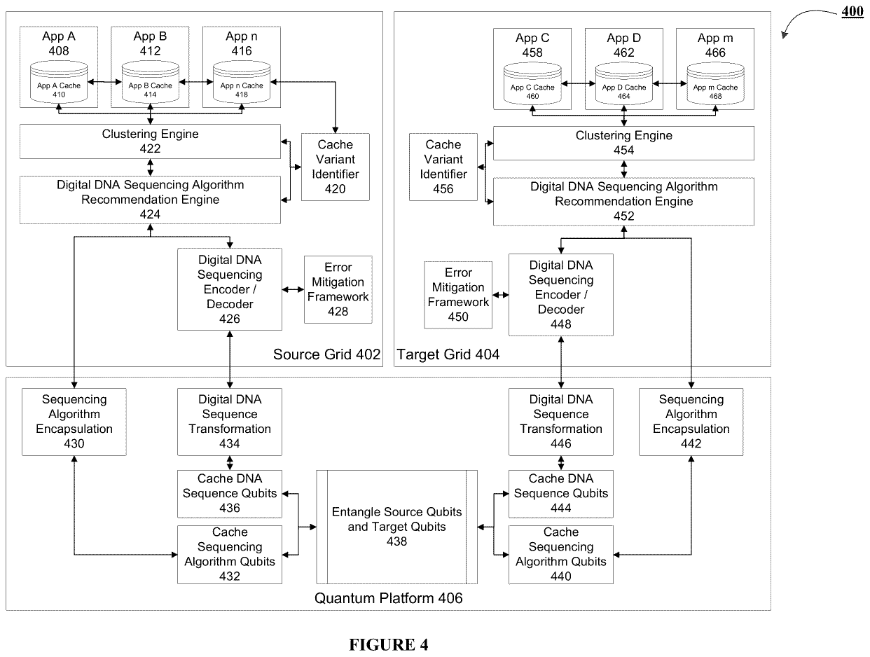

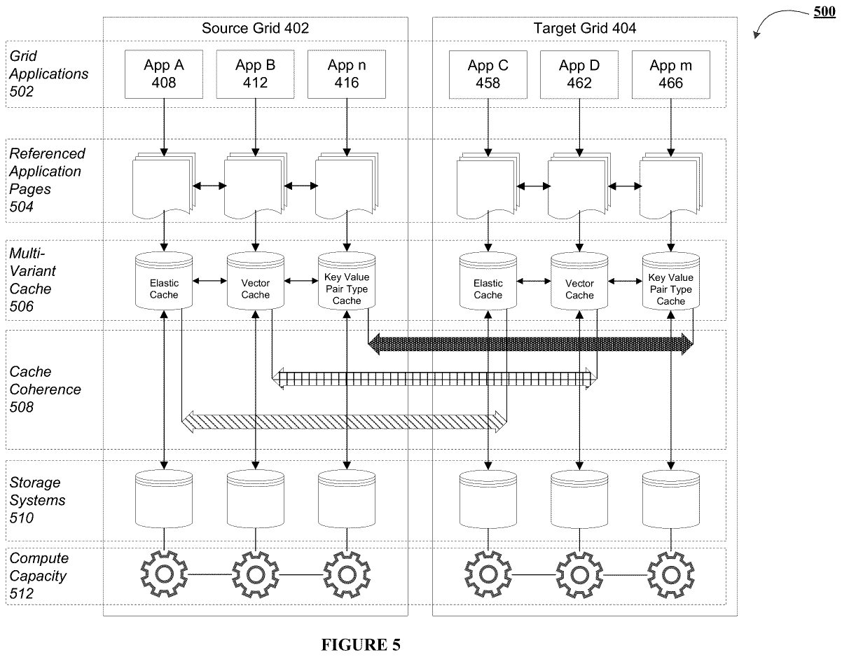

As shown in block 302, the process flow 300 of this embodiment includes receiving an update associated with cached data from a source application associated with a source grid. For example, as shown in FIG. 4, one or more applications (e.g., application A 408, application B 412, application n 416, etc.) may be associated with a source grid 402. Each of the applications may have their own cache. In some embodiments, the cache may be built-in cache specific to each of the applications. For example, application A 408 may have its own cache 410, application B 412 may have its own cache 414, and application n may have its own cache 418. In some embodiments, one or more of the caches 410, 414, 418, etc. may be updated. In this way, the update may include a configuration, re-configuration, or the like of the data in the cache. In some embodiments, the solutions as described herein may efficiently and effectively transmit the updated cache to one or more other caches associated with one or more other grids (e.g., the target grid 404). For example, with reference to FIG. 5, the grid applications 502 may include the applications associated with the source grid 402 and the target grid 404. In some embodiments, the target grid applications may receive the source grid application update.

In some embodiments, the cache may include cache data. The cache data may be multi-variant data, which may include one or more different file types. For example, the cache may include vector data, unstructured data, key-value pair data, and/or multi-media data (e.g., images, audio, text, video, etc.). In some embodiments, the multi-variant data may include data used by analytical or machine learning applications, logs or text documents, data within NoSQL databases, or data used by media-driven or content-rich applications. In some embodiments, and with reference to FIG. 5, the multi-variant cache 506 may also include reference application pages 504 where an application may reference one or more pages of other applications. For instance, application A 408 may reference pages or data associated with application B 412.

Further, as shown in block 304 of FIG. 3, the solution may include clustering the update based on chronological order in which the update was received. For example, as shown in FIG. 4, in some embodiments, the clustering engine 422 may cluster the data based on chronological order. In this way, the clustering engine 422 may cluster the cache data based on the order in which the data was received. Additionally, or alternatively, and in some embodiments, the clustered data may include various types of cache data. For example, the clustered data may include key-value pair data, unstructured data, and multimedia data.

In some embodiments, clustering the update based on chronological order in which the update was received may include configuring the update in chronological order to form the cluster, generating a unique identifier for the cluster, and assigning the unique identifier to the cluster. In some embodiments, the incoming cache updates or clusters may be assigned a timestamp that indicates the time at which the update was received. In this regard, the incoming updates (e.g., cached data updates or clustered updates) may be monitored for their respective timestamps as they arrive from their various applications (e.g., source applications). In some embodiments, the updates may be arranged or sorted in various manners, including but not limited to ascending order based on the timestamps, descending order based on timestamps, or the like. Further, in some embodiments, the clustered updates may be ordered based on a different ordering schema that may include ordering based on prioritization of the clustered update, for example.

In some embodiments, the clustered updates may be organized chronologically and grouped into a cluster, which may include a list, array, or other data structure that may represent the updates for further processing. In some embodiments, the solution may include generating the unique identifier associated with the clustered update, as shown in block 306 of FIG. 3. In some embodiments, the identifier may be generated to distinguish particular clusters from other clusters in the system. Further, in some embodiments, the identifier may contain identifying information associated with the cluster, such as embedded metadata, that may be used for indexing, lookup, communication services, or further processing such as encoding or verification.

In some embodiments, the unique identifier may include a cache variant identifier (e.g., the cache variant identifier 420 of FIG. 4), or a plurality of cached variant identifiers to identify the data types of the cached data. For example, the cache variant identifier 420 may be generated for the cache data. The cache variant identifier 420 may identify the type of data within the cache. For example, if application A's cache 410 contains key-value pair data, a cache variant identifier may be generated for that type of data. Further, in this example, if application B's cache 414 also contains key-value pair data, the cache variant identifier used to identify application A's cache 410 may also be used to identify application B's cache 414. In this way, and in some embodiments, the cache variant identifier 420 may be re-used to identify the same types of data throughout all of the applications associated with the source grid 402.

In other embodiments, the cache variant identifier 420 may be generated for each instance of data. For example, rather than re-using the cache variant identifier 420 for the same types of data across multiple applications, new cache variant identifiers may be generated for data from each application. Further, in some embodiments, the applications (408, 412, 416, etc.) may have multiple types of data within their respective caches. In this regard, multiple cache variant identifiers may be created for each type of data within the same application. For example, application A's cache 410 may have unstructured data, key-value pair data, and multimedia data, each of which may receive their own cache variant identifier 420.

Further, in some embodiments, the clustering engine 422 may be used to cluster the data received from the one or more applications (e.g., applications 408, 412, 416, etc.) based on the cache variant identifier 420. The clustering engine 422 may receive the data from the one or more applications'cache (e.g., application caches 410, 414, 418, etc.) and use the cache variant identifier 420 to cluster the data into clusters based on data type. In some embodiments, the clustered data may be the same type of data, as identified by the cache variant identifier 420. For example, all the key-value pair data from each of the applications 408, 412, and 416, as identified by the cache variant identifier 420, may be clustered by the clustering engine 422. In this way, the clustered data may include the cached and/or updated cached data from each of the applications'caches 410, 414, and 418.

In some embodiments, a digital DNA sequencing algorithm recommendation engine 424 may recommend a digital DNA sequencing algorithm used to create a digital DNA sequence of the clustered data. The digital DNA sequencing algorithm recommendation engine 424 may use a deep learning techniques to choose the best fit algorithm to be used for the digital DNA sequence creation. In addition, the recommendation engine may evaluate key parameters such as data type, size, redundancy requirements, expected error rates, and the like to determine which algorithm is best suited for digital DNA sequencing. In some embodiments, the most suitable DNA encoding technique may be chosen based on speed, data integrity, resource usage, compression ratios, error correction capabilities, or the like.

Further, in some embodiments, the digital DNA sequencing algorithm recommendation engine 424 may use the cache variant identifier 420 to determine which algorithm to use for the digital DNA sequence. In some embodiments, the deep learning engine may have the same or similar components as the machine learning model of FIG. 2. In this way, the deep learning engine may be trained, configured, and/or reconfigured based on new data ingested from the applications, the applications'caches, or the like.

As shown in block 308 of FIG. 3, the solution may include encoding, via a digital DNA encoder, the clustered update using a digital DNA sequencing algorithm into a single digital DNA sequence. In some embodiments, once one or more cache updates are aggregated into a cluster, the digital DNA encoder (e.g., digital DNA encoder 426) may apply a selected digital DNA sequencing algorithm to convert each data element into symbolic characters or tokens (e.g., A, C, G, T). In this regard, the characters or tokens (e.g., “nucleotides”) may be used to represent the underlying digital data (e.g., the cache data). Further, the system may map the data onto a set of four characters, for instance, instead of working with bits (0 and 1). In some embodiments, the consolidation of the cache data into a single digital DNA sequence may provide uniform representation for different file formats (e.g., vector data, unstructured data, key-value pair data, etc.) and streamline subsequent processing to promote interoperability across various grids (e.g., the source grid 402, the target grid 404, and the quantum platform 406). Additionally, or alternatively, many sequencing algorithms may incorporate compression features that reduce redundancy or network overhead in some cases. In this regard, relevant metadata may be embedded directly into the digital DNA sequence.

It is to be understood the characters or symbols as discussed herein are for exemplary purposes only and should be interpreted to limit the use of other symbols or characters used in the digital DNA sequence. For example, other symbols, characters, or tokens may be used in the digital DNA sequence that may be used as a universal transition layer that converts the cache data from diverse file formats into robust, compressed, and configurable representations of the data.

For example, in some embodiments, when the cached data updates are grouped into a cluster, the digital DNA encoder may apply the selected digital DNA sequencing algorithm (e.g., determined by a recommendation engine) to convert the cache data and/or multivariate cache data into a digital DNA sequence. In this example, the selected algorithm may translate the underlying data into characters or symbols (e.g., A, C, G, T). In this regard, the conversion of the data into a uniform representation may be easier to store, transmit, decode, and the like across multiple grids and platforms.

In some embodiments, the single digital DNA sequence may include a representation of the entire cluster of cache data. In some embodiments, the digital DNA sequence may be used to encode data in an arbitrary format into a standardized digital DNA format. For example, the digital DNA sequence may be used to capture key pieces of information such as the cache data updates, the unique identifier (e.g., cache variant identifier 420) associated with the cluster, and/or any necessary ordering structure associated with the sequence. Further, in some embodiments, the digital DNA sequence may represent a wide array of data types in a standardized format. Additionally, or alternatively, and in some embodiments, the digital DNA sequence may be format-agnostic and not rely on the data type of the cached data. In this way, the digital DNA sequencing algorithm may be chosen to best fit the type of cached data but the digital DNA sequence may be data agnostic.

In some embodiments, the digital DNA encoder may include error mitigation framework used to generate data that went missing during the encoding of the digital DNA sequence. In some embodiments, the error mitigation framework may include techniques that mitigate the errors caused during the digital DNA sequencing of the cached data. For example, computational errors that arise during encoding the digital DNA sequence may be limited by the error mitigation framework.

In some embodiments, encoding the clustered update using the digital DNA sequencing algorithm may include generating the single digital DNA sequence using a digital DNA sequencing algorithm recommendation engine comprising a ML engine that recommends the digital DNA sequencing algorithm. In some embodiments, the computational (e.g., digital DNA sequencing) methods used may analyze the cached data and determine which digital DNA sequencing algorithm is best suited for efficiently converting the cached data into the digital DNA sequence. In some embodiments, the algorithms may include the Overlap-Layout-Consensus (OLC) algorithm, the Fast Alignment using Sequences Through Translated Alignments (FASTA) algorithm, or the like.

In some embodiments, encoding the clustered update using the digital DNA sequencing algorithm may include generating the single digital DNA sequence using a digital DNA sequencing algorithm recommendation engine including a ML engine that recommends the digital DNA sequencing algorithm based on the plurality of cached variant identifiers.

As shown in block 310 of FIG. 3, the solution may include generating, via a quantum platform, a source qubit based on the single digital DNA sequence. In some embodiments, generating the source qubit may include converting the digital DNA sequence into one or more quantum qubits using an inter-transformation quantum processor module. For example, a Luby Transform along with error mitigation techniques in the quantum processor may be used to ensure the data is reliable. In some embodiments, the compressed data may be divided into small, fixed-sized segments (e.g., packets) which are encoded using random linear code generated by the Luby Transform. In some embodiments, the Luby Transform may ensure that the original data may be retrieved with high probability despite having some missing information, as long as enough good information is available. For example, with enough good packets remaining, the system may be able to retrieve the original data even if a certain number of packets were dropped or went missing during conversion.

In some embodiments, the source qubit may include at least two sets of qubits that may include at least a first qubit associated with the update associated with the cached data from the source application, and at least a second qubit associated with the digital DNA sequencing algorithm. In this regard, some qubits (e.g., the at least first qubit) may be used to capture the cached data converted into the digital DNA sequence. In some embodiments, the at least first qubits may be used to transmit the cache updates of the applications associated with the source grid. Further, in some embodiments, other qubits (e.g., the at least second qubit) may be used to capture the algorithm used to generate the digital DNA sequence. In this regard, the at least second qubit may be used during decoding to choose the correct decoding algorithm in the target grid.

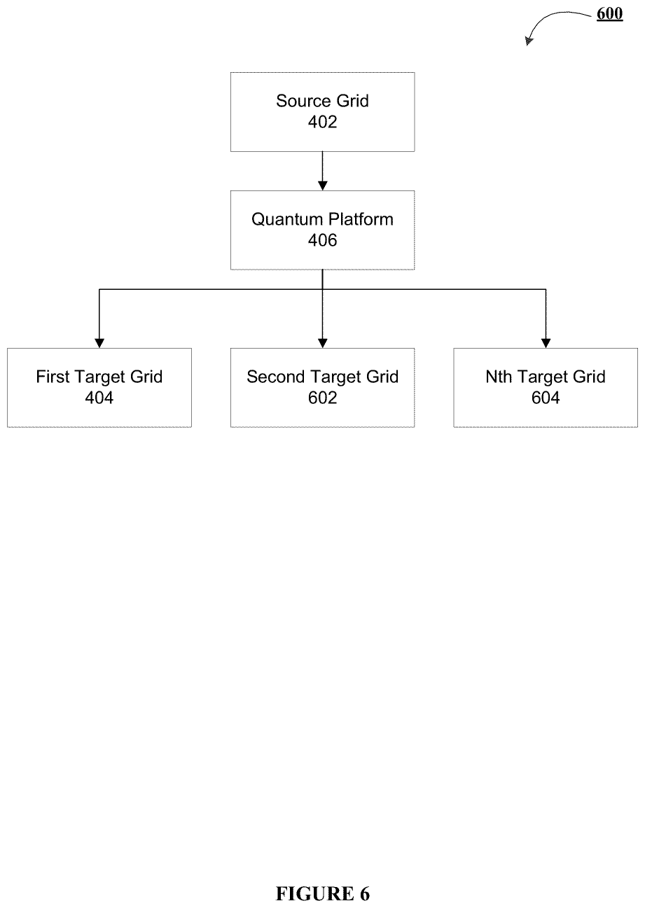

As shown in block 312 of FIG. 3, the solution may include configuring the source qubit using a GHZ quantum multipartite entanglement technique to entangle the source qubit with a target qubit associated with a target grid. In some embodiments, the multipartite entanglement technique may allow for the source grid qubits to be entangled with one or more target grid qubits. In this regard, the source grid may be used to transmit the cache updates to multiple target grids. For example, and as shown in FIG. 6, the source grid 402 may transfer its cache updates to a first target grid 404, a second target grid 602, an Nth target grid 604, and so on. Further, in some embodiments, the cache updates may be transmitted simultaneously from the source grid to the target grid(s).

In some embodiments, the GHZ entanglement technique may include preparing the qubits in a quantum state where all participant qubits (e.g., from the source grid and the target grid) share a single, cohesive quantum state. In this regard, measuring each qubit in the GHZ configuration reveals the interdependencies within the entire system. In the perspective of cache synchronization, once a clustered update is encoded into the source qubits, the entanglement technique may configure the target qubits to have the same resulting quantum state. Further, in some embodiments, the entanglement techniques may ensure that the target grid(s) receive the requisite information without the need for multiple, separate transmissions. In this regard, the GHZ multipartite entanglement technique promotes scalable, multi-target cache coherence which greatly enhances data synchronization across distributed computing environments.

As shown in block 314 of FIG. 3, the solution may include decoding, at the target grid and using a digital DNA decoder, the single digital DNA sequence received from the entangled qubits to retrieve the clustered update. For example, the digital DNA sequencing encoder/decoder 448 may decode the digital DNA sequence into the cache updates that may be used to update the caches (e.g., 460, 464, and/or 468) of the applications (e.g., 458, 462, and/or 466).

As shown in block 316 of FIG. 3, the solution may include configuring a target application's cached data with the clustered update. For example, as shown in FIG. 4, the applications associated with the target grid 404 (e.g., application C 458, application D 462, or application m 466) may have their caches (e.g., application C cache 460, application D cache 464, or application m cache 468) updated with the cache updates associated with the source grid 402. In some embodiments, after the digital DNA sequence has been converted into the cache data, the clustered update may be ready for integration into the caches. The cache updates may configure the target grid applications'caches (e.g., 458, 462, 466) to reflect the updates of the source grid applications'caches (e.g., 408, 412, 416).

Further, as shown in FIG. 5, the clustered update may configure the cache to which the cache data applies. For example, the source grid 402 may include an application cache associated with vector cache data, as shown in block 506. The vector cache update may configure an application's cache in the target grid 404 that is also associated with vector data. In this regard, the cache update may be transmitted to the appropriate application and/or application cache depending on the source formatting of the cache update. In some embodiments, the cache coherence 508 (e.g., maintaining the appropriate data format for each cache update) may apply for all application cache updates associated with the source grid 402 and target grid 404. Further, in some embodiments, the updates may be used for storage in storage systems 510 and/or further processing in computing resources (e.g., 512) of each respective grid.

In some embodiments, the target grid may include at least one of the same components as the source grid. For example, the target grids 404, 602, and 604 as shown in FIG. 6 may include components that allow the cache updates transfer to the target grids'target applications. Further, as shown in FIG. 4, the target grid 404 may include the same or similar components as the source grid 402. For example, the clustering engine 454 may perform the same or similar functionality as the clustering engine 422, the cache variant identifier 456 may perform the same or similar function as the cache variant identifier 420, the digital DNA sequencing algorithm recommendation engine 452 may perform the same or similar functionality as the digital DNA sequencing algorithm recommendation engine 424, the digital DNA sequencing encoder/decoder 448 may perform the same or similar functionality as the digital DNA sequencing encoder/decoder 426, and the error mitigation framework 450 may perform the same or similar functionality as the error mitigation framework 428. In some embodiments, the components of the target grid 404 may include the functionality to transmit one or more cache updates from its applications (e.g., applications 458, 462, and 466) and their caches 9 e.g., 460, 464, and 468) to the source grid 402 or other target grids. In this regard, the source grid 402, the target grid 404, and/or other target grids may include the ability to be a grid that either transmits or receives the cache updates of its applications.

As will be appreciated by one of ordinary skill in the art, the present disclosure may be embodied as an apparatus (including, for example, a system, a machine, a device, a computer program product, and/or the like), as a method (including, for example, a business process, a computer-implemented process, and/or the like), as a computer program product (including firmware, resident software, micro-code, and the like), or as any combination of the foregoing. Many modifications and other embodiments of the present disclosure set forth herein will come to mind to one skilled in the art to which these embodiments pertain having the benefit of the teachings presented in the foregoing descriptions and the associated drawings. Although the figures only show certain components of the methods and systems described herein, it is understood that various other components may also be part of the disclosures herein. In addition, the method described above may include fewer steps in some cases, while in other cases may include additional steps. Modifications to the steps of the method described above, in some cases, may be performed in any order and in any combination.

Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A system for configuring data in a distributed networking environment, the system comprising:
a processing device;
a non-transitory storage device containing instructions when executed by the processing device, causes the processing device to perform the steps of:
receive an update associated with cached data from a source application associated with a source grid;
cluster the update based on chronological order in which the update was received;
generate a unique identifier associated with the clustered update;
encode, via a digital DNA encoder, the clustered update using a digital DNA sequencing algorithm into a single digital DNA sequence;
generate, via a quantum platform, a source qubit based on the single digital DNA sequence;
configure the source qubit using a Greenberger-Horne-Zeilinger (GHZ) quantum multipartite entanglement technique to entangle the source qubit with a target qubit associated with a target grid;
decode, at the target grid and using a digital DNA decoder, the single digital DNA sequence received from the entangled qubits to retrieve the clustered update; and
configure a target application's cached data with the clustered update.

2. The system of claim 1, wherein clustering the update based on chronological order in which the update was received comprises:
configuring the update in chronological order to form the cluster;
generating a unique identifier for the cluster; and
assigning the unique identifier to the cluster.

3. The system of claim 1, wherein the target grid comprises at least one of the same components as the source grid.

4. The system of claim 1, wherein encoding the clustered update using the digital DNA sequencing algorithm comprises generating the single digital DNA sequence using a digital DNA sequencing algorithm recommendation engine comprising a machine learning (ML) engine that recommends the digital DNA sequencing algorithm.

5. The system of claim 1, wherein the cached data comprises multi-variant data including data types of at least vector data, unstructured data, key-value pair data, or multimedia data.

6. The system of claim 5, wherein a plurality of cached variant identifiers is used to identify the data types of the cached data.

7. The system of claim 6, wherein encoding the clustered update using the digital DNA sequencing algorithm comprises generating the single digital DNA sequence using a digital DNA sequencing algorithm recommendation engine comprising a ML engine that recommends the digital DNA sequencing algorithm based on the plurality of cached variant identifiers.

8. The system of claim 1, wherein the digital DNA encoder comprises error mitigation framework used to generate data that went missing during the encoding of the digital DNA sequence.

9. The system of claim 1, wherein the source qubit comprises at least two sets of qubits comprising:
at least a first qubit associated with the update associated with the cached data from the source application; and
at least a second qubit associated with the digital DNA sequencing algorithm.

10. A computer program product for configuring data in a distributed networking environment, the computer program product comprising a non-transitory computer-readable medium comprising code causing an apparatus to:
receive an update associated with cached data from a source application associated with a source grid;
cluster the update based on chronological order in which the update was received;
generate a unique identifier associated with the clustered update;
encode, via a digital DNA encoder, the clustered update using a digital DNA sequencing algorithm into a single digital DNA sequence;
generate, via a quantum platform, a source qubit based on the single digital DNA sequence;
configure the source qubit using a GHZ quantum multipartite entanglement technique to entangle the source qubit with a target qubit associated with a target grid;
decode, at the target grid and using a digital DNA decoder, the single digital DNA sequence received from the entangled qubits to retrieve the clustered update; and
configure a target application's cached data with the clustered update.

11. The computer program product of claim 10, wherein clustering the update based on chronological order in which the update was received comprises:
configuring the update in chronological order to form the cluster;
generating a unique identifier for the cluster; and
assigning the unique identifier to the cluster.

12. The computer program product of claim 10, wherein the target grid comprises at least one of the same components as the source grid.

13. The computer program product of claim 10, wherein encoding the clustered update using the digital DNA sequencing algorithm comprises generating the single digital DNA sequence using a digital DNA sequencing algorithm recommendation engine comprising a ML engine that recommends the digital DNA sequencing algorithm.

14. The computer program product of claim 10, wherein the cached data comprises multi-variant data including data types of at least vector data, unstructured data, key-value pair data, or multimedia data.

15. The computer program product of claim 14, wherein a plurality of cached variant identifiers is used to identify the data types of the cached data.

16. The computer program product of claim 15, wherein encoding the clustered update using the digital DNA sequencing algorithm comprises generating the single digital DNA sequence using a digital DNA sequencing algorithm recommendation engine comprising a ML engine that recommends the digital DNA sequencing algorithm based on the plurality of cached variant identifiers.

17. The computer program product of claim 10, wherein the digital DNA encoder comprises error mitigation framework used to generate data that went missing during the encoding of the digital DNA sequence.

18. The computer program product of claim 10, wherein the source qubit comprises at least two sets of qubits comprising:
at least a first qubit associated with the update associated with the cached data from the source application; and
at least a second qubit associated with the digital DNA sequencing algorithm.

19. A method for configuring data in a distributed networking environment, the method comprising:
receiving an update associated with cached data from a source application associated with a source grid;
clustering the update based on chronological order in which the update was received;
generating a unique identifier associated with the clustered update.
encoding, via a digital DNA encoder, the clustered update using a digital DNA sequencing algorithm into a single digital DNA sequence;
generating, via a quantum platform, a source qubit based on the single digital DNA sequence;
configuring the source qubit using a GHZ quantum multipartite entanglement technique to entangle the source qubit with a target qubit associated with a target grid;
decoding, at the target grid and using a digital DNA decoder, the single digital DNA sequence received from the entangled qubits to retrieve the clustered update; and
configuring a target application's cached data with the clustered update.

20. The method of claim 19, wherein clustering the update based on chronological order in which the update was received comprises:
configuring the update in chronological order to form the cluster;
generating a unique identifier for the cluster; and
assigning the unique identifier to the cluster.
